SEQUENCE LISTING (27 sequences)

SEQ ID NO: 1 - DNA, 408 bp, Mus sp.
atggcttgggtgtggaccttgctattcctgatggcagctgcccaaagtatccaagcacagatccagttggtgcagtctggacctgagttgaagaagcctggagagacagtcaagatctcctgcaaggcttctggatatactttcacaaactatggaatgaactgggtgaagcaggctccaggaaagggtttaaagtggatgggctggataaacaccgacagtggagagtcaacatatgctgaagagttcaagggacggtttgccttctctttggaaacctctgccaacactgcctatttgcagatcaacaacctcaacaatgaggacacggctacatatttctgtgtgagagtcggctacgatgctttggactactggggtcaaggaacctcagtcaccgtctcctca

SEQ ID NO: 2 - protein, 136 aa, Mus sp.
MAWVWTLLFLMAAAQSIQAQIQLVQSGPELKKPGETVKISCKASGYTFTNYGMNWVKQAPGKGLKWMGWINTDSGESTYAEEFKGRFAFSLETSANTAYLQINNLNNEDTATYFCVRVGYDALDYWGQGTSVTVSS

SEQ ID NO: 3 - DNA, 384 bp, Mus sp.
atggatttacaggtgcagattatcagcttcctgctaatcagtgcctcagtcataatgtccagaggacaaattgttctcacccagtctccagcaatcatgtctgcatctccaggggagaaggtcaccataacctgcagtgccaggtcaagtgtaagttacatgcactggttccagcagaagccaggcacttctcccaaactctggatttataggacatccaacctggcttctggagtccctgctcgcttcagtggcagtggatctgggacctcttactgtctcacaatcagccgaatggaggctgaagatgctgccacttattactgccagcaaaggagtagtttcccactcacgttcggctcggggacaaagttggaaataaaa

SEQ ID NO: 4 - protein, 128 aa, Mus sp.
MDLQVQIISFLLISASVIMSRGQIVLTQSPAIMSASPGEKVTITCSARSSVSYMHWFQQKPGTSPKLWIYRTSNLASGVPARFSGSGSGTSYCLTISRMEAEDAATYYCQQRSSFPLTFGSGTKLEIK

SEQ ID NO: 5 - protein, 23 aa, artificial sequence
  Frame region L1, derived from human TEL9 antibody
EIVLTQSPSSLSASVGDRVTITC

SEQ ID NO: 6 - protein, 15 aa, artificial sequence
  Frame region L2, derived from human TEL9 antibody
WXQQKPGKAPKLXIY

SEQ ID NO: 7 - protein, 32 aa, artificial sequence
  Frame region L3, derived from human TEL9 antibody
GVPSRFSGSGSGTXXXLTINSLQPEDFATYYC

SEQ ID NO: 8 - protein, 10 aa, artificial sequence
  Frame region L4, derived from human TEL9 antibody
FGGGTKLEIK

SEQ ID NO: 9 - protein, 30 aa, artificial sequence
  Frame region H1, derived from human hsighv1295 antibody
QXQLVQSGSELKKPGASVKISCKASGYXFX

SEQ ID NO: 10 - protein, 14 aa, artificial sequence
  Frame region H2, derived from human hsighv1295 antibody
WVXQAPGQGLXWMG

SEQ ID NO: 11 - protein, 32 aa, artificial sequence
  Frame region H3, derived from human hsighv1295 antibody
RFXFSLDTSVXTAYLQITSLXAEDTGMYFCXX

SEQ ID NO: 12 - protein, 11 aa, artificial sequence
  Frame region H4, derived from human hsighv1295 antibody
WGQGTLVTVSS

SEQ ID NO: 13 - protein, 10 aa, Artificial Sequence
  Synthetic peptide derived from murine BAT antibody
SARSSVSYMH

SEQ ID NO: 14 - protein, 7 aa, Artificial Sequence
  Synthetic peptide derived from murine BAT antibody
RTSNLAS

SEQ ID NO: 15 - protein, 9 aa, Artificial Sequence
  Synthetic peptide derived from murine BAT antibody
QQRSSFPLT

SEQ ID NO: 16 - protein, 5 aa, Artificial Sequence
  Synthetic peptide derived from the CDR of the heavy cahin variable region of murine BAT antibody
NYGMN

SEQ ID NO: 17 - protein, 17 aa, Artificial Sequence
  Synthetic peptide derived from the CDR of the heavy cahin variable region of murine BAT antibody
WINTDSGESTYAEEFKG

SEQ ID NO: 18 - protein, 8 aa, Artificial Sequence
  Synthetic peptide derived from the CDR of the heavy cahin variable region of murine BAT antibody
VGYDALDY

SEQ ID NO: 19 - protein, 106 aa, Artificial Sequence
  Synthetic peptide corresponding to one alternative of the light chain variable region of humanized BAT
EIVLTQSPSSLSASVGDRVTITCSARSSVSYMHWYQQKPGKAPKLLIYRTSNLASGVPSRFSGSGSGTDFTLTINSLQPEDFATYYCQQRSSFPLTFGGGTKLEIK

SEQ ID NO: 20 - protein, 106 aa, Artificial Sequence
  Synthetic peptide  corresponding to one alternative of the light chain variable region of humanized BAT
EIVLTQSPSSLSASVGDRVTITCSARSSVSYMHWFQQKPGKAPKLWIYRTSNLASGVPSRFSGSGSGTDYTLTINSLQPEDFATYYCQQRSSFPLTFGGGTKLEIK

SEQ ID NO: 21 - protein, 106 aa, Artificial Sequence
  Synthetic peptide  corresponding to one alternative of the light chain variable region of humanized BAT
EIVLTQSPSSLSASVGDRVTITCSARSSVSYMHWFQQKPGKAPKLWIYRTSNLASGVPSRFSGSGSGTDYCLTINSLQPEDFATYYCQQRSSFPLTFGGGTKLEIK

SEQ ID NO: 22 - protein, 106 aa, Artificial Sequence; identical to SEQ ID NO: 21
  Synthetic peptide  corresponding to one alternative of the light chain variable region of humanized BAT

SEQ ID NO: 23 - protein, 117 aa, Artificial Sequence
  Synthetic peptide  corresponding to one alternative of the heavy chain variable region of humanized BAT
QVQLVQSGSELKKPGASVKISCKASGYSFSNYGMNWVRQAPGQGLQWMGWINTDSGESTYAEEFKGRFVFSLDTSVSTAYLQITSLTAEDTGMYFCAKVGYDALDYWGQGTLVTVSS

SEQ ID NO: 24 - protein, 117 aa, Artificial Sequence
  Synthetic peptide  corresponding to one alternative of the heavy chain variable region of humanized BAT
QVQLVQSGSELKKPGASVKISCKASGYTFTNYGMNWVRQAPGQGLQWMGWINTDSGESTYAEEFKGRFVFSLDTSVSTAYLQITSLTAEDTGMYFCAKVGYDALDYWGQGTLVTVSS

SEQ ID NO: 25 - protein, 117 aa, Artificial Sequence
  Synthetic peptide  corresponding to one alternative of the heavy chain variable region of humanized BAT
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 26 - protein, 117 aa, Artificial Sequence
  Synthetic peptide  corresponding to one alternative of the heavy chain variable region of humanized BAT
QIQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 27 - protein, 117 aa, Artificial Sequence
  Synthetic peptide  corresponding to one alternative of the heavy chain variable region of humanized BAT
QIQLVQSGSELKKPGASVKISCKASGYTFTNYGMNWVKQAPGQGLKWMGWINTDSGESTYAEEFKGRFAFSLDTSVNTAYLQITSLNAEDTGMYFCVRVGYDALDYWGQGTLVTVSS

(19) United States
(12) Patent Application Publication
(10) Pub. No.: US 2009/0123413 A1
(43) Pub. Date: May 14, 2009

(54) USE OF BAT MONOCLONAL ANTIBODY FOR IMMUNOTHERAPY

(76) Inventors: Britta Hardy, Tel Aviv (IL); Annat Raiter, Kfar Saba (IL); Michael Schickler, Mazkeret Batya (IL)

Correspondence Address: WINSTON & STRAWN LLP;PATENT DEPARTMENT, 1700 K STREET, N.W., WASHINGTON, DC, 20006, US

(21) Appl. No.: 11/721,307

(22) PCT Filed: Aug. 23, 2005
(86) PCT No.: PCT/IL05/00909

§ 371 Date: Jun. 8, 2007

Related U.S. Application Data
(60) Provisional application No. 60/603,273, filed on Aug. 23, 2004.

(51) Int. Cl. A61K 39/395; A61K 38/21; A61K 38/20

(52) U.S. Cl. 424/85.2; 424/152.1; 424/136.1; 424/133.1; 424/135.1; 424/85.5; 424/85.7

(57) ABSTRACT

The present invention relates to immunotherapy and more specifically concerns the use of immunostimulatory BAT monoclonal antibodies for treatment of a variety of immunodeficiency related diseases and disorders and malfunction or incompetence of the immune system.

### FIELD OF THE INVENTION

The present invention relates to immunotherapy and more specifically concerns the use of immunostimulatory BAT monoclonal antibodies for treatment of a variety of immuno-deficiency related diseases and disorders and malfunction or incompetence of the immune system.

### BACKGROUND OF THE INVENTION

Cancer immunotherapeutics are aimed by and large at modulating the response of the immune system to induce or enhance killing of tumor cells. This approach utilizes using various immunomodulators including monoclonal antibodies that selectively bind to a specific determinant on T cells thereby either initiating an activation pathway or inducing an inhibitory effect (Kohler, G. and Milstein, C., 1995, Nature 256:495).

The main tumor-cell-killing effector cells are cytotoxic T lymphocytes, though accumulating evidence suggests that T-helper cells are also essential for priming the immune system against tumors (Pardoll, D. M. and Topalian, S. L., 1998, Cur. Opinions Immunol. 10:588). T-helper cells activate nonspecific immune effector mechanisms in the course of tumor destruction by secreting appropriate cytokines such as interferon-gamma among others (Sadanaga, N. et al., 1999, J. Immunother., 22:315).

BAT is a monoclonal antibody that was generated against a membrane preparation of a Burkitt lymphoma cell line (Daudi) and exhibits anti-tumor and immunostimulatory effects towards various types of tumors (Hardy et al., 2001, Int. J. Oncol. 19:897). BAT monoclonal antibody was initially disclosed in U.S. Pat. No. 5,897,862 to Hardy et al., an inventor of the present invention, which is incorporated in its entirety by reference, as is fully set forth herein.

The polynucleotide and amino-acid sequences of murine BAT are disclosed in WO 00/58363, to Hardy et al., and US Patent Publication No. 20030026800, both publications incorporated herein by reference.

A humanized monoclonal BAT antibody is disclosed in WO 03/099196 to Hardy et al., which is incorporated in its entirety herein by reference. The humanized monoclonal BAT antibody appears to induce a greater anti-tumor effect than those induced by the parent murine BAT antibody.

Among other model systems tested, the BAT anti-tumor activity was studied in SCID (severe combined immunodeficiency disease) mice, beige mice that are deficient in NK cells and nude mice that are deficient in T cells. (Hardy, B., 1997, Proc. Natl. Acad. Sci. USA 94:5756). All mice were injected i.v. with murine B16 melanoma that subsequently develops tumors in the lungs. BAT exerted an anti-tumor effect only in SCID mice that were engrafted with either murine or human lymphocytes. In the athymic nude mice and the beige mice BAT exerted an anti-tumor activity, though this activity was less effective as compared to the anti-tumor activity of BAT in the wild-type mice.

The immunomodulatory effect of BAT was studied also in vitro. Murine BAT activates CD4⁺ T cells and induces the secretion of IFN-γ from these cells (Hardy et al., 2000, Int. Immunol. 12:1623 and Quaglino E. et al., 2005, Vaccine 9:23(25):3280-7, respectively). In addition, Hardy et al. showed that BAT triggers the proliferation of T cells and increases their cytolytic activity (Hardy, B. et al., 1997, Hum. Antibodies, 8:95).

Nowhere in the background art is the use of BAT monoclonal antibody for immunotherapy or immunostimulation of immunodeficient patients, including AIDS or SCID patients, taught or suggested. There is an unmet need for compositions and methods for the treatment of immunodeficiency disorders, depletion of lymphocytes, reduction in lymphocyte number and malfunction of lymphocytes.

### SUMMARY OF THE INVENTION

In accordance with some aspects of the present invention, the BAT monoclonal antibody is disclosed as a therapeutic agent for the treatment of immunodeficiency disorders, including, but not limited to, severe combined immunodeficiency disease, acquired immunodeficiency syndrome, and any disorder that involves depletion, attenuation and/or malfunctioning of lymphocytes, specifically T cells, NK cells, NK-T cells, B cells, monocytes, macrophages or any combination thereof. The present invention further discloses use of BAT monoclonal antibody for the treatment of immunodeficiency, immune malfunction or immune incompetence, collectively referred to hereinafter as immunodeficiency disorders, established after chemotherapy or irradiation. According to certain embodiments, BAT monoclonal antibody is used in conjunction with autologous, allogeneic or syngeneic stem cell transplantation derived from the bone marrow, umbilical cord blood or peripheral blood and donor leukocyte infusion. In particular, the BAT monoclonal antibody, including any variation comprising at least the antigen binding portion thereof is now disclosed as a therapeutic agent for induction of lymphopoiesis, as well as for expansion, proliferation, activation and differentiation of lymphocytes.

According to one aspect, the present invention provides a method for treating an immunodeficiency disorder in a subject in need thereof, comprising administering a therapeutically effective amount of BAT monoclonal antibody to the subject.

According to a preferred embodiment, the subject in need thereof is a human subject.

The present invention is based in part on the unexpected finding that BAT exerts a significant lymphopoietic activity, in vivo, when injected into naïve or tumor-bearing nude mice, as disclosed in Hardy et al. (International Immunology, 17:615-619, Mar. 31, 2005), which was published after the priority date of the present application and is incorporated herein in its entirety by reference. Moreover, the lymphopoietic effect of BAT is surprisingly significant not only in tumor-bearing mice but even in naïve mice that do not bear tumors. The present invention is also based on the unexpected finding that administration of humanized BAT (also termed hereinafter CT-011) to patients suffering from advanced stages of hematological malignancies that were previously treated with chemotherapy, irradiation therapy, transplantation of stem cells derived from either the bone marrow, umbilical cord blood or peripheral blood or donor leukocyte infusion (DLI) resulted in significant increase in the percentage of peripheral CD4 T cells within about 24 hr post administration of the humanized BAT.

According to another aspect, the present invention provides use of BAT in the preparation of a medicament for treating an immunodeficiency disorder. According to certain embodiments, the BAT monoclonal antibody comprises a light chain variable region comprising CDRs selected from the group consisting of: SEQ. ID NO. 13; SEQ. ID NO. 14 and SEQ. ID NO. 15. According to further embodiments, the BAT monoclonal antibody comprises a heavy chain variable region comprising CDRs selected from the group consisting of: SEQ. ID NO. 16; SEQ. ID NO. 17 and SEQ. ID NO. 18.

According to one embodiment, the immunodeficiency disorder comprises at least one of the disorders selected from the group consisting of: depletion of lymphocytes, reduction in lymphocyte number and malfunction of lymphocytes.

According to another embodiment, the lymphocytes include at least one population of cells selected from the group consisting of: NK-cells, NK-T cells, B cells, T-cells, CD3⁺ cells, CD4⁺ cells, CD8⁺ cells, Thy1.2+ cells subpopulations thereof and a combination thereof.

According to yet another embodiment, the immunodeficiency disorder is congenital. According to yet another embodiment, the immunodeficiency disorder is acquired.

According to yet another embodiment, the immunodeficiency disorder is selected from the group consisting of: severe combined immunodeficiency disease, acquired immunodeficiency syndrome, X-linked agammaglobulinaemia, common variable immunodeficiency, IgA deficiency, IgG subclass deficiency, Wiskott-Aldrich syndrome, DiGeorge anomaly, Ataxia Telangiectasia, adenosine deaminase deficiency and activation-induced cytidine deaminase deficiency.

According to yet another embodiment, the immunodeficiency disorder is related to viral infection, fungal infection or bacterial infection. According to yet another embodiment, the immunodeficiency disorder is associated with intoxication.

According to yet another embodiment, the method of the invention is used for treating anemia, particularly, aplastic anemia and Myelodysplastic syndromes (MDS), primarily for avoiding further complication of the anemia due to immunodeficiency disorders.

According to yet another embodiment, the immunodeficiency disorder is associated with any one of the treatments selected from: chemotherapy, irradiation, transplantation of stem cells derived from either the bone marrow, umbilical cord blood or peripheral blood and donor leukocyte infusion. According to yet another embodiment the bone marrow transplantation is autologous, syngeneic or allogeneic.

According to various specific embodiments, the BAT monoclonal antibody is selected from the group consisting of: full length monoclonal antibody, chimeric antibody, humanized antibody, IgG, IgM, IgD, IgA, IgE, diabody, bispecific antibody, linear antibody and fragments thereof.

According to yet another embodiment, the BAT monoclonal is a humanized antibody wherein the frame regions of the light chain variable region are derived from the light chain variable region of the human TEL9 antibody. According to yet another embodiment, said frame regions are selected from the group consisting of: SEQ. ID NO. 5; SEQ. ID NO. 6; SEQ. ID NO. 7 and SEQ. ID NO. 8.

According to yet another embodiment, the BAT monoclonal is a humanized antibody wherein the frame regions of the heavy chain variable region are derived from the heavy chain variable region of the human hsighv1295 antibody. According to yet another embodiment, said frame regions are selected from the group consisting of: SEQ. ID NO. 9; SEQ. ID NO. 10; SEQ. ID NO. 11 and SEQ. ID NO. 12.

According to yet another embodiment, the antibody fragment is selected from the group consisting of: Fab, Fab′, F(ab′)₂, Fv; single-chain antibody molecules and multi-specific antibodies formed from antibody fragments.

According to yet another embodiment, the method for treating an immunodeficiency disorder in a subject in need thereof, further comprises administering at least one additional therapeutic agent in combination with a therapeutically effective amount of BAT monoclonal antibody, the at least one additional therapeutic agent being selected from the group consisting of: anti-viral agents, antibiotics, cytokines, T-cell activators, hormones, growth factors (e.g. GM-CSF), cell vaccines, peptide vaccines, DNA vaccines, antibodies and fragments thereof and T-cell stimulatory antibodies.

According to yet another embodiment, the method further comprises administering at least one additional anti-cancer agent. According to yet another embodiment the at least one additional anti-cancer agent is selected from: anti-metabolic agent, anti-angiogenic agents, cytotoxic agents and anti-tumor therapeutic antibodies.

According to yet another embodiment, the T-cell activators are selected from the group consisting of: interleukin-1 (IL-1), interleukin-2 (IL-2) interleukin-4 (IL-4), interleukin-5 (IL-5), interleukin-6 (IL-6), interleukin-7 (IL-7), interleukin-12 (IL-12), interleukin-13 (IL-13), interleukin-15 (IL-15), interferon-alpha (IFNα), interferon-gamma (IFNγ), tumor necrosis factors such as TNFα, anti-CD3 antibodies (anti-CD3), anti-CD28 antibodies (anti-CD28), anti-CTLA4 antibodies, anti-TGF-beta antibodies, anti-4-1BB antibodies, cell-based vaccines peptide vaccines, DNA vaccines, growth factors, phytohemagglutinin, concanavalin-A and phorbol esters.

According to some embodiments, the lymphocyte activators are produced by any one of the methods selected from a group consisting of: derivation from a natural source, production by recombinant DNA technology and chemical synthesis.

According to yet another embodiment, said therapeutically effective amount of BAT monoclonal antibody and said at least one therapeutic agent are administered together or sequentially.

Other objects, features and advantages of the present invention will become clear from the following description and drawings.

### BRIEF DESCRIPTION OF THE FIGURES

FIG. 1 shows a FACS dot plot analysis of CD4 and CD8 T cell sub-populations (A-B and C-D, respectively) in blood of nude mice treated with mouse IgG3 (A and C) or BAT (B and D).

FIG. 2 demonstrates a FACS dot plot analysis of CD3/Thy-1.2 (A-B), CD4/Thy-1.2 (C-D) T cell sub-populations in blood of nude mice treated with mouse IgG3 (A and C) or BAT (B and D).

FIG. 3 presents histograms obtained from FACS analysis of CD3, CD4 and CD56 leukocytes (panels A-C, respectively) drawn from the blood of naïve nude mice 17 days following BAT administration (thin line), from the blood of naYve nude mice 17 days post control IgG3 administration (thick line) or from the blood of nude mice inoculated with HM7 tumor for 17 days and treated with BAT for 5 days (broken line)

### DETAILED DESCRIPTION OF THE INVENTION

The present invention provides, by a first of its aspects, a method for the treatment of immunodeficiency disorders in a subject in need of such treatment comprising administering a therapeutically effective amount of BAT, so as to thereby treat the subject.

As use herein, the term “BAT” is used in a broad sense and specifically covers a monoclonal antibody or an antigen binding fragment thereof, wherein the monoclonal antibody is secreted by the hybridoma cell line deposited at the Collection Nationale de Cultures de Microorganismes (CNCM), under Accession No. 1-1397, or recognizes the same antigenic epitope as the antibody in the above, as fully disclosed in U.S. Pat. No. 5,897,862 herein incorporated by reference in its entirety including supplements; A monoclonal antibody as fully described in U.S. Patent Application Publication No. 20030026800 (incorporated herein in its entirety by reference including supplements), A humanized monoclonal antibody, as fully described in WO03/099196 which is incorporated herein by reference in its entirety.

Thus, according to certain embodiments, the BAT monoclonal antibody is characterized as follows: an antibody having a heavy chain variable region encoded by the polynucleotide sequence set forth in SEQ ID NO. 1; an antibody having a heavy chain variable region as set forth in SEQ ID NO:2; an antibody having a light chain variable region encoded by the polynucleotide sequence set forth in SEQ ID NO. 3 and/or an antibody having a light chain variable region as set forth in SEQ ID NO:4, as disclosed in U.S. Patent Application Publication No. 20030026800.

According to other embodiments, the BAT monoclonal antibody is a humanized antibody having a light chain variable region characterized by the formula:

FR_{L1}-CDR_{L1}-FR_{L2}-CDR_{L2}-FR_{L3}-CDR_{L3}-FR_{L4}

wherein the FRs are derived from the light chain variable region of the human TEL9 antibody and selected from the group consisting of: FR_{L1}, [EIVLT QSPSS LSASV GDRVT ITC; SEQ. ID NO. 5]; FRL2, [W (F or Y) QQKPG KAPKL (W or L) IY; SEQ. ID NO. 6]; FRL3, [GVPSR FSGSG SGT (D or S) (Y or F) (C or T) LTINS LQPED FATYY C; SEQ. ID NO. 7]; FRL4, [FGGGT KLEIK; SEQ. ID NO. 8]

and having a heavy chain variable region characterized by the formula:

FR_{H1}-CDR_{H1}-FR_{H2}-CDR_{H2}-FR_{H3}-CDR_{H3}-FR_{H4}

wherein the FRs are derived from the heavy chain variable region of the human hsigv1295 antibody and selected from the group consisting of: FR_{H1}, [Q (I or V) QLV QSGSE LKKPG ASVKI SCKAS GY (T or S) F (T or S); SEQ. ID NO. 9]; FR_{H2}, [WV (R or K) QAPGQ GL (Q or K) WMG; SEQ. ID NO. 10]; FR_{H3}, [RF (V or A) FSLDT SV (N or S) TAYLQ ITSL (T or N) AEDTG MYFC (V or A) (R or K); SEQ. ID NO. 11]; FRH4, [WGQGT-LVTVS S; SEQ. ID NO. 12]

and wherein the CDRs are derived from the murine BAT-1 antibody (the subscripts “L” and “H” refer to light and heavy chain regions, respectively) and are selected from the group consisting of: CDR_{L1} [SARSS VSYMH; SEQ. ID NO. 13]; CDR_{L2} [RTSNL AS; SEQ. ID NO. 14]; CDR_{L3} [QQRSS FPLT; SEQ. ID NO. 15]; CDR_{H1} [NYGMN; SEQ. ID NO. 16]; CDR_{H2} [WINTD SGEST YAEEF KG; SEQ. ID NO. 17]; CDR_{H3} [VGYDA LDY; SEQ. ID NO. 18], as disclosed in International Patent Application, Publication No. WO03/099196.

Human TEL-9 antibody was identified in diverse libraries of immunoglobulin heavy (VH) and light (V kappa and V lambda) chain variable (V) genes prepared from peripheral blood lymphocytes of unimmunized donors (Marks et al. J Mol Biol. 1991, 222:581-97). This antibody was showin to bind specifically to the turkey egg-white lysozyme (TEL) antigen. Human hsiggv1295 antibody was isolated from stable hybridomas and Epstein-Barr virus-transformed B cell lines from the synovial fluid or peripheral blood of three patients with rheumatoid arthritis and one patient with systemic lupus erythematosus (Fang et al., J Exp Med. 1994, 179:1445-56).

According to particular embodiments, the light chain variable region of the humanized BAT monoclonal antibody is a selected from the group consisting of: BATR_{κa} (SEQ. ID NO. 19), BATR_{κb} (SEQ. ID NO. 20), BATR_{κc} (SEQ. ID NO. 21), BATR_{κd} (SEQ. ID NO. 22) an the heavy chain variable region is selected from the group consisting of: BATRH_{A} (SEQ. ID NO. 23), BATRH_{B} (SEQ. ID NO. 24), BATRH_{C} (SEQ. ID NO. 25), BATRH_{D} (SEQ. ID NO. 26) or BATRH_{E} (SEQ. ID NO. 27).

According to some embodiments, the humanized monoclonal antibody comprises a variable region selected from the group consisting of: BATRH_{A/}BATR_{κa} (SEQ. ID NO. 23/SEQ. ID NO. 19), BATRH_{B}/BATR_{κa} (SEQ. ID NO. 24/SEQ. ID NO. 19), BATRH_{B}/BATR_{κb} (SEQ. ID NO. 24/SEQ. ID NO. 20), BATRH_{C}/BATR_{κb} (SEQ. ID NO. 25/SEQ. ID NO. 20), BATRH_{B}/BATR_{κd} (SEQ. ID NO. 24/SEQ. ID NO. 22), or BATRH_{C}/BATR_{κd} (SEQ. ID NO. 25/SEQ. ID NO. 22).

The term “antibody” is used in the broadest sense and specifically covers monoclonal antibodies (including full length monoclonal antibodies) of any of the classes IgG, IgM, IgD, IgA, IgE and antibody fragments so long as they exhibit the desired biological activity. “Antibody fragments” comprise a portion of a full-length antibody, generally the antigen binding or variable region thereof. Examples of antibody fragments include Fab, Fab′, F(ab′)₂, and Fv fragments; diabodies; linear antibodies; single-chain antibody molecules; and multi-specific antibodies formed from antibody fragments.

The term “monoclonal antibody” as used herein refers to antibodies that are highly specific, being directed against a single antigenic epitope. Alternatively, the term “monoclonal antibody” as used herein refers to an antibody produced from a single spleen cell clone. The monoclonal antibodies to be used in accordance with the present invention may be made by recombinant DNA methods (see, e.g., U.S. Pat. Nos. 5,225,539 to Winter and 5,530,101 to Queen et al.).

In accordance with one embodiment of the invention, the monoclonal antibody is a chimeric human-mouse antibody, namely a mAb with a constant region derived from a human origin and a variable region derived from mouse. For this purpose, the Kappa light and heavy chain variable regions of the mAb of the invention were PCR cloned and their DNA sequenced.

In accordance with yet another embodiment of the invention the antibody is a fully humanized antibody, i.e. both its variable and constant region are derived from a human source.

In accordance with various specific embodiments, the BAT monoclonal antibody is selected from the group consisting of: full length monoclonal antibody, chimeric antibody, humanized antibody, IgG, IgM, IgD, IgA, IgE, diabody; linear antibody and fragments thereof.

In accordance with yet various specific embodiments, the antibody fragment is selected from the group consisting of: Fab, Fab′, F(ab′)₂, Fv; single-chain antibody molecules and multi-specific antibodies formed from antibody fragments.

The term “diabody” refers to a dimeric antibody fragment. In each polypeptide, a heavy-chain variable domain (VH) is linked to a light-chain variable domain (VL) but unlike single-chain Fv fragments, each antigen-binding site is formed by pairing of one VH and one VL domain from the two different polypeptides. Diabodies thus have two antigen-binding sites, and can be bispecific.

The term “bispecific antibody” refers to an antibody that is able to specifically bind to two different molecules. Binding of a bispecific antibody to a first molecule takes place with one F(ab′) binding arm, and binding to a second molecule, such as a tumor-specific antigen on a tumor cell, takes place with another F(ab′) binding arm. A bispecific antibody may further bind an FcR via its Fc portion.

In accordance with one another embodiment of the invention, the provided method is used to treat any immunodeficiency disorder, resulting from intoxication or chemotherapy and/or irradiation and/or related to therapeutic procedures involving transplantation of stem cells derived from either the bone marrow, umbilical cord blood or peripheral blood or donor leukocyte infusion that involves depletion, reduction in number, abnormal or malfunction of lymphocytes, including, but not limited to, T cells, NK cells, B cells, monocytes, macrophages or any combination thereof.

The term “T cell” as used herein refers to a lymphocyte that matures in the thymus and expresses a T-cell receptor, CD3, and CD4 or CD8 or Thy1.2 thymocytes. This term encompasses T cell subpopulations and combinations thereof.

The term “natural killer” or “NK” cell as used herein refers to a large, granular lymphocyte that has a cytotoxic ability but does not express antigen-binding receptors. The term “NK-T” as used herein refers to CD3 lymphocytes that present NK cell markers. The term “B cell” as used herein refers to antibody-producing lymphocyte lineage. The term “monocytes” as used herein refers to myeloid lineage cells. The term “macrophages” as used herein refers to differentiated monocytes.

In accordance with another embodiment of the invention, a method for treating congenital immunodeficiency disorder as well as acquired immunodeficiency disorder is provided. The immunodeficiency disorder includes, for example, a severe combined immunodeficiency disease (SCID), acquired immunodeficiency syndrome (AIDS), X-linked agammaglobulinaemia, common variable immunodeficiency, IgA deficiency, IgG subclass deficiency, Wiskott-Aldrich syndrome, DiGeorge anomaly, Ataxia Telangiectasia (A-T), adenosine deaminase deficiency (ADA), activation-induced cytidine deaminase deficiency (AICDA or AID deficiency). The immunodeficiency disorder maybe associated with earlier or an ongoing treatment such as chemotherapy, irradiation, autologous, allogeneic or syngeneic transplantation of stem cells derived from the bone marrow, umbilical cord blood or peripheral blood and donor leukocyte infusion.

Although Ataxia Telangiectasia is not an immunodeficiency disorder per se, this disease is associated with variable immunologic diseases and immunologic deficits, which primarily lead to recurrent pneumonia, bronchiectases and chronic obstructive and restrictive pulmonary disease. Thus, including administration of BAT antibody in the treatment regimen of Ataxia Telangiectasia, is mostly directed to treat the immunologic deficits associated with Ataxia Telangiectasia.

In some other embodiments of the invention, the method is used for treating immune deficiency and/or malfunction due to any type of chemotherapy whether combined or not with radiation therapy. The method of the invention may be further used for treating immunodeficiency disorder related to viral infection, fungal infection or bacterial infection and for treating immunodeficiency disorders, which are associated with intoxication, including, but not limited to intoxication as a result of chemotherapy and chemical intoxication among others.

In yet other embodiments, the method for treating an immunodeficiency disorder in a subject in need thereof, further comprises administering at least one additional therapeutic agent in combination with a therapeutically effective amount of BAT monoclonal antibody, the at least one additional therapeutic agent being selected from the group consisting of: anti-viral agents, antibiotics, cytokines, T-cell activators, hormones, growth factors, cell vaccines, peptide vaccines, DNA vaccines, antibodies and fragments thereof and T-cell stimulatory antibodies.

In an alternative embodiment, the method of the invention further comprises administering at least one additional anti-cancer agent selected from: anti-metabolic agent, anti-angiogenic agents, cytotoxic agents and anti-tumor therapeutic antibodies.

In yet other embodiments of the invention, the method is used for treating immune deficiency and/or malfunction due to any type of bacterial, fungal or viral infection.

Immunodeficiency disorders may evolve from malfunction of the immune system, leading to the development of infections that recur frequently, are more severe, and last longer than usual. Immunodeficiency disorders impair the ability of the immune system to defend the body against invasions and attacks by foreign or abnormal cells (such as bacteria, viruses, fungi, and cancer cells). As a result, infections and cancers may develop.

Immunodeficiency disorders may be present at birth (congenital, or primary) or may develop later in life, often as a result of another disorder (acquired, or secondary). Congenital immunodeficiency disorders are usually hereditary. They typically become evident during infancy or childhood. There are more than 70 congenital immunodeficiency disorders; all are relatively rare. Acquired immunodeficiency disorders are much more common. Some immunodeficiency disorders shorten lifespan, others persist throughout life but do not affect lifespan, and a few resolve with or without treatment.

Immunodeficiency disorders are grouped by which part of the immune system is affected. They may involve problems with antibodies, T lymphocytes, both B and T lymphocytes, phagocytes, or complement proteins. The affected component of the immune system may be: depletion of T-cells, reduction in T-cell number and/or malfunction of T-cells.

Severe combined immunodeficiency disease (SCID) is considered as the most serious immunodeficiency disorder. It is a congenital immunodeficiency disorder resulting in low levels of antibodies and a low number and malfunction of T lymphocytes. SCID can be caused by several different genetic defects, most of which are hereditary. Most infants with severe combined immunodeficiency disease develop pneumonia, thrush, and diarrhea, usually by age 3 months. More serious infections, including pneumocystis pneumonia, can also develop. If not treated, these children usually die before age 2. The common treatment to date is by antibiotics and immune globulin or transplantation of stem cells from bone marrow or umbilical cord blood.

HIV infection is a viral infection that progressively destroys the white blood cells and causes acquired immunodeficiency syndrome (AIDS). The two human immunodeficiency viruses HIV-1 and HIV-2 progressively destroy lymphocytes, which are an important part of the body's immune defenses, thereby, turning the body much more susceptible to attack by many other infectious organisms. Many of the symptoms and complications of HIV infection, including death, are the result of these other infections and not of the HIV infection itself. HIV infection may lead to various troublesome infections with organisms that do not ordinarily infect healthy people (opportunistic infections).

Acquired immunodeficiency syndrome (AIDS) is considered as the most severe form of HIV infection. Because the number of CD4⁺ lymphocytes in the blood helps determine the ability of the immune system to protect the body from infections, it is a good measure of the severity of the damage done by HIV infection. A healthy person has a CD4⁺ lymphocyte count of roughly 800 to 1,300 cells per microliter of blood. Typically, 40 to 60% of CD4⁺ lymphocytes are destroyed in the first few months of infection. After about 6 months, the CD4⁺ count stops falling so quickly, but it continues to decline. If the CD4⁺ count falls below about 200 cells per microliter of blood, the immune system becomes less able to fight certain infections. A count below about 50 cells per microliter of blood is particularly dangerous, because additional opportunistic infections that can rapidly cause severe weight loss, blindness, or death commonly occur.

AIDS has been directly linked to an increased incidence of malignancies. Kaposi's sarcoma, non-Hodgkin's lymphoma, and cervical cancer are AIDS-defining illnesses in HIV-infected patients. Other neoplastic diseases associated with AIDS include Hodgkin's disease, anal cancer, testicular cancer, melanoma, nonmelanomatous skin cancers, lung cancer, and primary CNS lymphoma. Leiomyosarcoma has been reported as a rare complication of HIV infection in children.

In one embodiment, the immunodeficiency and/or malfunction or incompetence of the immune system is a consequence of chemotherapy and/or irradiation and/or intoxication. According to yet another embodiment the immunodeficiency and/or malfunction or incompetence of the immune system is associated with treatment procedures involving autologous and/or syngeneic and/or allogeneic transplantation of stem cells derived from either the bone marrow, umbilical cord blood or peripheral blood and/or donor leukocyte infusion (DLI).

Cancer patients are frequently treated with chemotherapy and irradiation. For some malignant diseases, especially hematological malignancies, therapy includes also autologous or syngeneic or allogeneic bone marrow transplantation or donor leukocyte infusion (DLI). These therapeutic interventions may result in transient or persistent complete or partial immune deficiency or immune malfunction, turning the body much more susceptible to attacks by infectious organisms. Successful cell-based therapy frequently results in enhanced anti tumor immune response and reduced susceptibility to infections.

Cell-based therapy as used herein may include lymphocyte infusions (donor leukocyte infusions), transplantation of stem cells derived from the bone marrow, umbilical cord blood or peripheral blood (autologous, syngeneic or allogeneic), dendritic cell based therapies or T cell based therapies and any combination thereof.

The present invention is directed however to any of the aforementioned immunodeficiency disorders of the immune system including improper immune responses against invading microorganisms (immunodeficiency disorders) among others.

In one embodiment, the method of treatment also comprises administration of BAT antibody or a composition comprising thereof to a subject in parallel to, prior to, or following treatment with an additional active composition comprising cytokines, lymphocyte activators, growth factors, mitogenic factors and other antibodies, such as any T-cell stimulatory antibody.

The growth factors and lymphocyte activators may include, without limitation, one or more of the following agents: interleukin-1 (IL-1), interleukin-2 (IL-2) interleukin-4 (IL-4), interleukin-5 (IL-5), interleukin-6 (IL-6), interleukin-7 (IL-7), interleukin-11 (IL-11), interleukin-12 (IL-12), interleukin-13 (IL-13), interleukin-15 (IL-15), interferon-alpha (IFNα), interferon-gamma (IFNγ), tumor necrosis factors such as TNFα, anti-CD3 antibodies (anti-CD3), anti-CD28 antibodies (anti-CD28), anti-CTLA4 antibodies, anti-TGF-beta antibodies, anti-4-1BB antibodies, cell-based vaccines comprising of inactivated or otherwise modified tumor or naive cells, peptide vaccines corresponding to cellular proteins whether tumor associated or not and any parts thereof, DNA vaccines encoding for cellular proteins whether tumor associated or not and any parts thereof, growth factors like erythropoietin or granulocyte-macrophage colony stimulating factor, mitogenic factors like phytohemagglutinin, concanavalin-A and phorbol esters. The lymphocyte activators, growth factors and mitogenic factors can be native factors obtained from natural sources, factors produced by recombinant DNA technology, chemically synthesized polypeptides or other molecules, or any derivative having the functional activity of the native factor.

In a currently preferred embodiment, the subject in need thereof is a human subject.

The present invention also provides use of BAT in the preparation of a medicament for therapy of an immunodeficiency disorder and/or malfunction or incompetence of the immune system. The immunodeficiency disorder may be any disorder that involves T-cell depletion, reduction in T-cell number, T-cell abnormalities and/or malfunction of T cell and/or NK cells and/or NK-T cells and/or B cells and/or monocytes and/or macrophages congenital or acquired and may include any disorder that mentioned above. Malfunction or incompetence of the immune system may be a consequence of chemotherapy, irradiation and/or intoxication.

In yet another preferred embodiment, the present invention also provides use of BAT in the preparation of a medicament for therapy in conjunction with procedures involving transplantation of stem cells derived from either the bone marrow, umbilical cord blood or peripheral blood and/or donor leukocyte infusion.

The present invention also involves use of a pharmaceutical composition for treating an immunodeficiency disorder, wherein the pharmaceutical active ingredient is BAT in a therapeutically effective amount. The pharmaceutical composition may further comprise a pharmaceutically acceptable carrier. Said composition may be in any pharmaceutical form suitable for administration to a patient, including but not limited to solutions, suspensions, lyophilized powders for reconstitution with a suitable vehicle or dilution prior to usage, capsules and tablets.

The administration of the compositions of the present invention can be typically achieved by means of parenteral administration, e.g., intravenously (i.v.) intraperitoneally (i.p.) subcutaneously (s.c.) or intramuscularly (i.m.) intradermally (i.d.). Methods of treatment may comprise pharmaceutical compositions of BAT according to the invention.

Preferably, the composition of the present invention has a form suitable for injections. The pharmaceutical composition disclosed in this invention may further comprise any pharmaceutically acceptable diluent or carrier to provide a physiologically acceptable conjugates comprising BAT with one or more therapeutic agents.

The pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes. Preferably, the pharmaceutical compositions of the present invention may be manufactured by processes that are particularly suitable for proteins, more particularly antibodies.

Pharmaceutical compositions for use in accordance with the present invention may be formulated in conventional manner using one or more physiologically acceptable carriers comprising excipients and auxiliaries, which facilitate processing of the active compounds into preparations and that are pharmaceutically suitable. Proper formulation is dependent upon the route of administration chosen.

For injection, the compounds of the invention may be formulated in aqueous solutions, preferably in physiologically compatible buffers such as Hank's solution, Ringer's solution, or physiological saline buffer.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art. Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile pyrogen-free water, before use.

Pharmaceutical compositions for use in accordance with the present invention may be formulated for delivery via inhalation in a manner using one or more physiologically acceptable carriers comprising excipients and auxiliaries, which facilitate processing of the active compounds into preparations and that are pharmaceutically suitable.

Pharmaceutical compositions suitable for use in context of the present invention include compositions wherein the active ingredient is contained in an amount effective to achieve the intended purpose. All formulations for administration should be in dosages suitable for the chosen route of administration. More specifically, a “therapeutically effective” dose means an amount of a compound effective to prevent, alleviate or ameliorate symptoms of a disease of the subject being treated. Determination of a therapeutically effective amount is well within the capability of those skilled in the art, especially in light of the detailed disclosure provided herein.

Toxicity and therapeutic efficacy of the compositions described herein can be determined by standard pharmaceutical procedures in cell cultures or experimental animals, e.g., by determining the IC₅₀ (the concentration which provides 50% inhibition) and the maximal tolerated dose for a subject compound. The data obtained from these cell culture assays and animal studies can be used in formulating a range of dosage for use in human. The dosage may vary depending inter alia upon the dosage form employed, the dosing regimen chosen, the composition of the agents used for the treatment and the route of administration utilized among other relevant factors. The exact formulation, route of administration and dosage can be chosen by the individual physician in view of the patient's condition. Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

In one embodiment of the invention, the effect of the composition may be the induction of lymphopoiesis and/or proliferation, activation or differentiation of cells, such as, T cells, NK cells, NK-T cells, Thy1.2 cells, B cells, monocytes, macrophages, transplanted stem cells derived from either the bone marrow, umbilical cord blood or peripheral blood or lymphocytes following donor leukocyte infusion.

In accordance with a certain embodiment, the BAT monoclonal antibody, being useful for a variety of therapeutic indications, is used for the treatment of immunodeficiency disorder.

In one embodiment, methods are provided for the use of a humanized BAT for the treatment of immunodeficiency disorder by administering to a subject an effective amount of the antibody of the invention. Since the antibodies used in such embodiment shall be used in an in vivo therapy, preferably antibodies or derivatives or fragments of human origin are used, or antibodies modified to be suitable for the use in humans (so-called “humanized antibodies”) (see for example U.S. Pat. Nos. 5,585,089 to Queen et al. and 5,225,539 to Winter).”

The term “effective amount” should be understood as meaning an amount of an antibody required to achieve a therapeutic effect. The effective amount required to achieve the therapeutic end result may depend on a number of factors including, for example, the specific type of the disorder and the severity of the patient's condition, and whether the antibody is co-administered together with another agent which acts together with the antibody in an additive or synergistic manner. BAT may be administered either following detection of a disorder in the subject or, as preventive therapy of a subject having a high risk of developing immunodeficiency disorder.

The dose of the composition to be administrated to a subject, in the context of the present invention should be sufficient to effect a beneficial therapeutic response in the subject over time, or to induce lymphopoiesis and/or proliferation, activation or differentiation of T cells, NK cells, NK-T cells, and B cells.

The dose will be determined by the activity of the therapeutic composition produced and the condition of the subject, as well as the body weight or surface area of the subject to be treated. The size of the dose and the dosing regiment also will be determined by the existence, nature, and extent of any adverse side effects that accompany the administration of a particular therapeutic composition in a particular subject. In determining the effective amount of the therapeutic composition to be administered, the physician needs to evaluate inter alia circulating plasma levels, toxicity, and progression of the disease.

Having now generally described the invention, the same will be more readily understood through reference to the following examples, which are provided by way of illustration and are not intended to be limiting of the present invention.

### EXAMPLES

### Materials and Methods

### Molecular Biology Techniques and CDR Grafting

Molecular biology techniques and CDR grafting protocols suitable to carrying out the invention as herein described are known to those skilled in the art. Suitable teachings are described in numerous manuals and primary publications, including inter alia, Sambrook et al., (Molecular Cloning: A Laboratory Manual, 2nd Ed., Cold Spring Harbor Laboratory Press, Cold Spring Harbor, N.Y., 1989); Ausubel et al., (Protocols In Molecular Biology, Green Publishing Associates and Wiley-Interscience, John Wiley and Sons, New York 1987, 1988, 1989); U.S. Pat. Nos. 5,225,539 to Winter and 5,585,089 to Queen et al., which are herein incorporated by reference in their entirety including supplements.

### PCR Technology

The term “Polymerase Chain Reaction” (“PCR”) refers to the methods disclosed in U.S. Pat. Nos. 4,683,195; 4,683,202, and 4,965,188 and improvements thereto.

### BAT Antibody

BAT monoclonal antibodies were selected by binding to Daudi cells and by their ability to induce human peripheral blood mononuclear cell proliferation (Hardy, et al., 1994. Cancer Res. 54:5793; Hardy, et al., 1989. Cell. Immunol. 118:22). The BAT monoclonal antibody may be produced from either in vitro hybridoma cultures grown in RPMI-1640 medium supplemented with 10% fetal calf serum (FCS) or from mice ascites, followed by purification on a protein G Sepharose column (Pharmacia, Uppsala, Sweden). Additionally, the BAT antibody may be obtained or produced in any method previously described by the inventors in U.S. Pat. No. 5,897,862; U.S. Pat. Application 20030026800; and WO03/099196 incorporated herein by reference in their entirety.

### Human Tumor Cell Line

HM7 is a sub-clone of the human CRC cell line LS174T, selected for its high mucin synthesis and metastatic potential (Bresalier, R. S., Niv, Y., Byrd, J. C., Duh, Q. Y., Toribara, N. W., Rockwell, R. W., Dahiya, R. and Kim, Y. S. 1991. Mucin production by human colonic carcinoma cells correlates with their metastatic potential in animal models of colon cancer metastasis. J. Clin. Invest. 87:1037). Cells were obtained from Professor Robert S. Breselier (MD Andersen, USA). The cells were grown in RPMI-1640 supplemented with 10% FCS, L-glutamine (2 mM), Na-pyruvate (1 nMol), penicillin (100 units/ml), streptomycin sulfate (0.1 mg/ml) and nystatine (12.5u/ml). Cultures were maintained at 37° C. in a humidified 5% CO₂ incubator.

### Human Colon Carcinoma Tumor Model in Nude Mice

A liver metastases human colon carcinoma tumor model was used in nude mice (Bresalier, R. S., Niv, Y., Byrd, J. C., Duh, Q. Y., Toribara, N. W., Rockwell, R. W., Dahiya, R. and Kim, Y. S. 1991. Mucin production by human colonic carcinoma cells correlates with their metastatic potential in animal models of colon cancer metastasis. J. Clin. Invest. 87:1037). BALB/c nude mice were anesthetized and their spleens were exposed. HM7 (2×10⁶) cells in 0.250 ml PBS were injected into the exposed spleen; after 1 minute, the spleens were removed and the excisions closed. HM7 cells colonize the liver as bulky nodules. Mice were divided into four groups: (1) Nude mice injected with BAT only (10 μg/mouse in PBS); (2) Nude mice injected with the isotype control mouse IgG3; (3) Nude mice injected with tumor cells only; and (4) Nude mice inoculated with tumor cells and 12 days post-tumor inoculation injected intravenously with BAT antibodies at 10 μg/mouse in PBS.

### Collecting Blood Samples of Nude Mice

Two blood sampling periods, “first” and “second” sampling, were defined equivalently across all four groups detailed above, based on corresponding times after BAT (or BAT control) injection. Thus, blood from groups 1 and 2 of nude mice was collected on day 5 (first sampling) and 14 (second sampling) post-antibody and control IgG injections. Blood from group 3 and 4 was collected 17 (first sampling) and 26 (second sampling) days post-tumor inoculation, corresponding to 5 and 14 days post-BAT injection (BAT administration on day 12 post-tumor inoculation, plus either 5 or 14 days).

### Fluorocytometric Cell Analysis of Lymphocytes in Blood

Mononuclear cells were isolated from the blood by Ficoll-Hypaque centrifugation. Thereafter, 5×10⁵ fresh isolated leukocytes samples were suspended in 50 μl PBS containing 5% FCS and 0.1% Na-azide. Cells were stained with saturated amounts of the indicated antibodies for 45 minutes on ice: Rat anti mouse-CD3 Pycoerythrin (PE) labeled (clone number KT3), rat anti mouse-CD4 (clone number YTS 191.1), anti mouse CD56 antibody, rat anti mouse CD8 (clone number KT15) fluorescein labeled (serotec, Oxford, UK) and anti mouse Thy1.2 (CD90.2) labeled with biotin (clone number 30-H12, BioLegend, San Diego, USA) followed by streptavidin Cy5 coupled to R-phycoerytrin (DAKO Cytomation). Triple staining dot blot analysis was performed using a FACScan (Beckton Dickinson, USA). PE-conjugated anti-NK1.1 (clone number PK136) antibodies were used in double staining with anti mouse CD3 labeled with FITC. Anti-mouse-kappa for detection of kappa positive B cells was labeled with biotin (Southern Biotechnology Associates, Birmingham, Ala.) and detected with streptavidin—FITC (Jackson Immuno-research, West Grove, Palo Alto, Calif.).

Side-scatter and forward-scatter of dot plots were used to determine the gates of lymphocytes; PE or FITC labeled IgGs (Pharmingen, San Diego, CS, USA) served as isotope controls for PE or FITC labeled antibodies. FACS analysis was done using FACSCalibur flow cytometer (Becton Dickinson, Esembodegem, Belgium). Data were analyzed using CELLQuest (Becton Dickinson, Erembodegem, Belgium).

### Example 1

### Effect of BAT Injection to Naive Nude Mice on Lymphocyte Populations in the Blood

The proportion of lymphocyte sub-populations in blood of nude mice was followed 5 days after a single I.V. injection of 10 μg/mouse of BAT antibody. FACS analysis of a representative experiment (Table 1) determined the percent of CD3, CD4, CD8 T cells, the proportion of NK1.1 positive NK cells and the percent of Thy1.2, Thy1.2/CD3. and Thy1.2/CD4 cells. Study of lymphocytes obtained from blood of control nude mice demonstrated a low proportion of CD3 and CD4 positive T cells, which varied between 0.1-0.6% 5 days post-control IgG3 administration. However, 5 days after BAT injection, the proportion of CD3-positive cells increased to 24.5% and the proportion of CD4 cells increased to 20.8% (p<0.05). Double staining of CD3/CD4 populations increased to 19.3%. In contrast to CD4 cell propagation, CD8 T cells in the blood of the nude mice did not increase at any tested day following the injection of BAT antibodies and the values were as low as in the controls (0.3-0.6%).

The number of NK1.1 positive NK cells, increased from 7.3% in the control to 18.5% after 5 days following BAT administration. BAT administration did not induce an increase in the percentage of Thy1.2 cells. However, BAT induced an increase in the percentage of CD3/Thy1.2 from 0.4% to 24% and CD4/Thy1.2 from 0.1% to 21%-(Table 1). FACS analysis of percent of CD3, CD4, CD8, Thy1.2 positive T cells and NK1.1 positive NK cells in blood of nude mice at different days post-injection of 10 ug/mouse of BAT antibody or mouse IgG3 isotype control is shown in Table 1 below:

TABLE 1
Percent of cells determined by FACS analysis

PERCENTAGE OF CELLS

| T-cell subtype | Control | BAT |
|---|---|---|
| CD3 | 0.8 | 24.5 |
| CD4 | 0.1 | 20.8 |
| CD3/CD4 | 0.1 | 19.3 |
| CD8 | 0.6 | 0.3 |
| CD3/CD8 | 0.3 | 0.3 |
| Thy1.2 | 29.6 | 30.2 |
| CD3/Thy1.2 | 0.4 | 24.0 |
| CD4/Thy1.2 | 0.1 | 20.9 |
| NK1.1 | 7.3 | 18.5 |

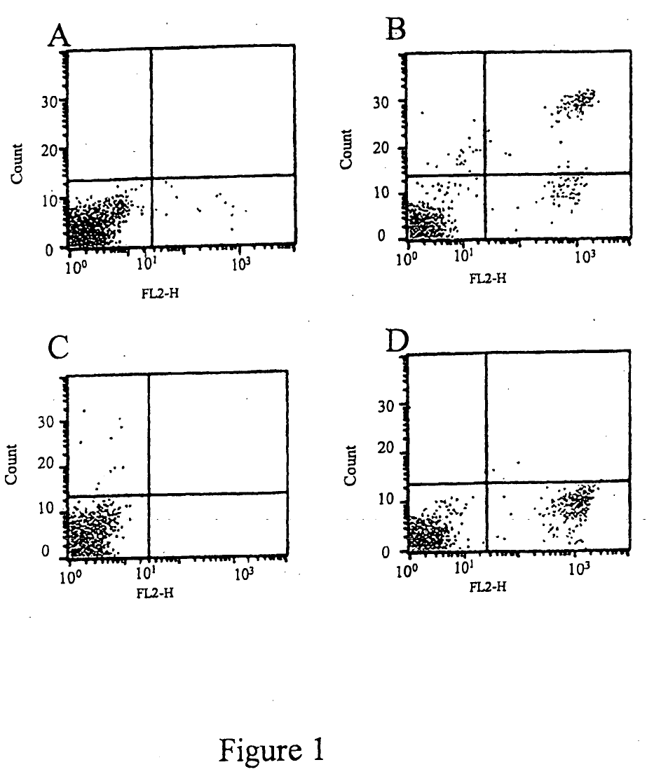

CD4 cells lymphopoiesis is induced 5 days post BAT administration, as depicted in FIG. 1. Lymphocytes from nude mice that were treated with or without BAT for 5 days, were stained with PE-labeled anti mouse CD3 (demonstrated by FL2-H staining, A-D) and with either FITC-labeled anti mouse CD4 (A-B), or FITC-labeled anti mouse CD8 (C-D). Acquisition of 10:000 gated cells was confirmed by CD3-labeled cells. Results of double staining CD3/CD4 and CD3/CD8 lymphocyte subpopulations indicate that BAT induces lymphopoiesis of CD3/CD4 cells and not of CD3/CD8 cells following 5 days of treatment.

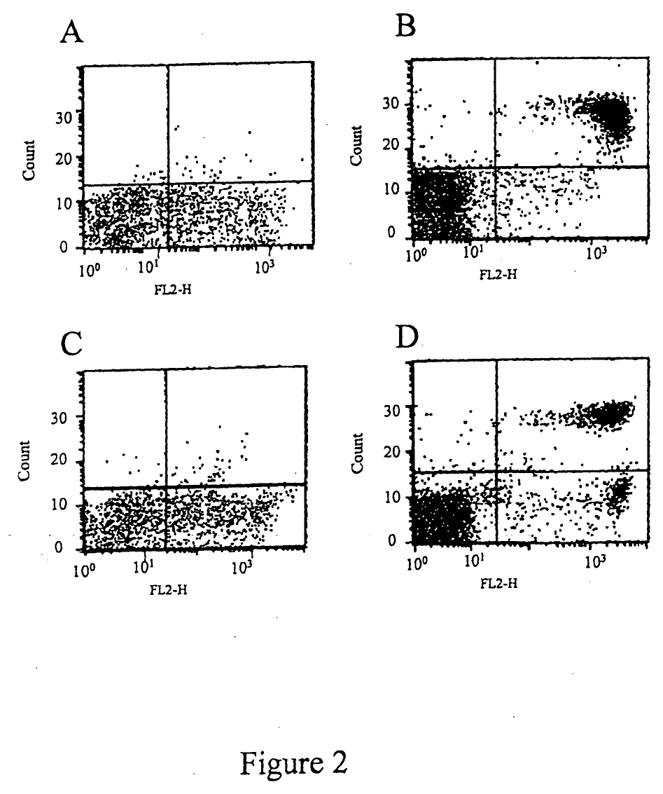

Increased lymphopoiesis of CD4 cells following BAT treatment is also demonstrated in FIG. 2. Lymphocytes from nude mice that were treated with or without BAT for 5 days, were stained with anti mouse biotin-labeled Thy1.2 antibody followed by streptavidin-RPE-Cy5 (demonstrated by FL3-H staining, A-D) and with either anti-mouse CD3 PE-labeled antibody (A-B), or with anti CD4 FITC labeled antibody (C-D). The results demonstrate that BAT mAb administration induces a distinct increase in CD3/Thy1.2 and CD4/Thy1.2 cells following 5 days of treatment.

The proportion of lymphocyte sub-populations in blood of nude mice was followed 5 and 14 days after a single injection with 10 μg/mouse of BAT antibody. FACS analysis determined the percent of CD3, CD4, CD8 T cells and proportion of CD56 positive cells NK. Lymphocytes in blood of control nude mice tested at the 2 time points maintained a low proportion of CD3 and CD4 positive T cells, which varied between 0.6 to 3 percent. However, 5 days after BAT injection, CD3 cells increased to 12 percent and reached 19 percent on day 14. CD4 cells increased after 5 days to 18.8% (p<0.05) and at 14 days settled at 14.6 percent. In contrast to CD4 cell propagation, CD8 T cells in the blood of the nude mice did not increase at any tested day following the injection of BAT antibodies and the values were as low as in the controls (0.1-1.1%)

The number of CD56 positive NK cells, increased from 2.6% in control to 25% 5 days post BAT administration and remained 24.2% 14 days post BAT treatment.

FACS analysis of percent of CD3, CD4, CD8 positive T cells and CD56 positive NK cells in blood of nude mice at different days post-injection of 10 ug/mouse of BAT antibody or mouse IgG3 isotype control is shown in Table 2 below:

TABLE 2
Percent of cells determined by FACS analysis

| | Time post treatment | |
|---|---|---|
| T-cell | Day 5 | Day 14 |

| subtype | Control | BAT | Control | BAT |
|---|---|---|---|---|
| CD3 | 1.2 | 12.0 | 3.0 | 19.0 |
| CD4 | 0.6 | 18.8 | 11.1 | 14.6 |
| CD8 | 0.0 | 1.1 | 0.0 | 0.0 |
| CD56 | 2.6 | 25.0 | 0.3 | 24.2 |

### Example 2

### Effect of BAT Treatment on Peripheral Blood Leukocytes in Nude Mice Engrafted with Human Colon Carcinoma

### A. Effect of HM7 Human Colon Carcinoma Engraftment of Nude Mice on Lymphocyte Populations in Blood

Percent of lymphocyte sub-populations in blood of nude mice engrafted with tumor was followed on the first (17 days post tumor inoculation) and second (26 days post tumor inoculation) samplings. The effect of tumor on blood lymphocytes determined by FACS analysis is presented in Table 3. As can be seen the percent of CD3, CD4, CD8 T cells was low and not significantly changed by tumor implantation: 1.2 to 3.5 percent for CD3, 1.1 and 2.7 for CD4 and 0.3 and 1.1 for CD8 cells. The number of anti-kappa positive B cells increased with time. NK cells were 7.4 on first sampling and 0.3 percent on second sampling.

Effect of human colon carcinoma HM7 administration on the percent of CD3, CD4, CD8 positive T cells, CD56 positive NK cells and B kappa positive B cells in the blood of nude mice is shown in Table 3 below:

TABLE 3
Percent of lymphocyte sub-populations in
blood of nude mice engrafted with tumors

Days post-tumor administration

| Lymphocyte sub-population | 17 days | 26 days |
|---|---|---|
| CD3 | 1.2 | 3.5 |
| CD4 | 1.1 | 2.7 |
| CD8 | 0.3 | 1.1 |
| anti-kappa | 52.7 | 87.4 |
| CD56 | 7.4 | 0.3 |

### B. Effect of BAT Treatment of Human Colon Carcinoma of Nude Mice on Peripheral Blood Leukocytes

Treatment of H-M7 tumor bearing mice with a single administration of BAT (10 ug/mouse) 12 days post-tumor administration induced a remarkable anti-tumor activity, manifested as prevention of liver metastases. Blood analysis (Table 4) tested on the first sampling (5 days post-BAT injection) demonstrated a significant 33.6% increase in CD3 cells (p<0.05) and 20.8% in CD4 positive cells (p<0.05). A similar effect of BAT treatment was also seen in increased number of NK cells on the first sampling (22.1%) and CD8 cells (9.7%). B cells bearing kappa-light chain were present in all groups of mice and were not significantly affected by BAT treatment (52.8%). On second sampling (14 days after BAT treatment, 26 days after tumor inoculation), CD3, CD4 and CD8 and NK cell subpopulations decreased to the control nude mice values in the blood.

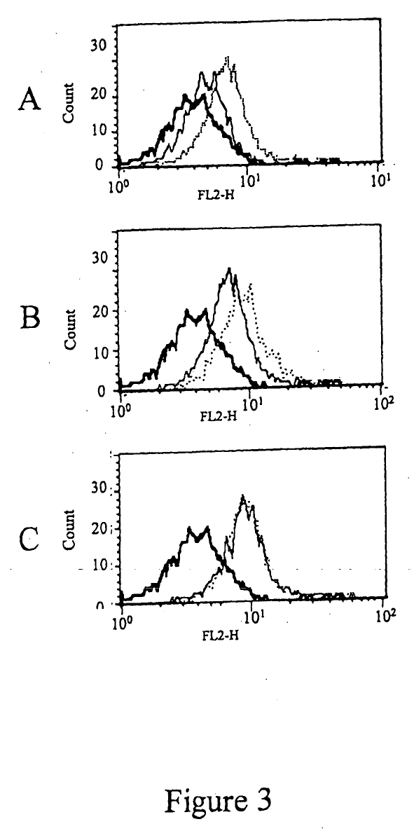

As can be seen in the FACS analysis shown in FIG. 3, (and also in table 2 and 4), the effect of BAT in tumor bearing mice is further enhanced compared to BAT stimulation of CD3 and CD4 in the non tumor bearing mice. The percentage of NK cells is enormously enhanced already 14 days after BAT treatment.

TABLE 4
Percent of lymphocytes in blood of nude mice injected
with BAT antibody post-human colon tumor in nude mice

| | 17 days after tumor | 26 days after tumor |
|---|---|---|
| | inoculation and 5 days | inoculation and 14 days |
| Lymphocyte subtypes | after BAT injection | after BAT injection |
| CD3 | 33.6 | 3.6 |
| CD4 | 20.8 | 9.2 |
| CD8 | 9.7 | 2.9 |
| Anti-kappa | 52.8 | 59.8 |
| CD56 | 22.1 | 0.7 |

### Example 3

### Treatment of Patients Suffering from Hematological Malignancies with Humanized BAT (CT-011 Antibody) Results in Significant Increase in Peripheral CD4 T Cells

Phase I clinical study was conducted for evaluating the safety of escalating doses of the humanized BAT (CT-011) administered to patients suffering from advanced stages of a variety of hematological malignancies i.e., AML (Acute myeloid leukemia), HD (Hodgkin's disease), NHL (Non Hodgkin's lymphoma), and CLL (Chronic lymphocytic leukemia). Medical history of these patients included chemotherapy and/or irradiation and/or autologous or allogeneic bone marrow transplantation and/or donor leukocyte infusion (DLI). Changes in blood lymphocytes were monitored by staining of peripheral blood lymphocytes drawn from said patients before and 24 hours after administration of 0.6 or 3 mg/kg CT-011 with labeled-anti human CD4 antibody followed by FACS analysis.

The results indicate significant increases in the percentage of peripheral CD4 T cells 24 hours post antibody administration from 22% to 34% (p=0.02) in patients treated with 0.6 mg/kg of CT-011 and from 33% to 42% (p=0.005) in patients treated with 3 mg/kg of CT-011 (Table 5).

TABLE 5
Effect of CT-011 on the percentage of CD4 T cells in patients
with advanced stage hematological malignancies

| | |
|---|---|
| | % of CD4 T cells (Mean ± SDV) |
| Phase of | CT-011 Dose (mg/kg) |

| Treatment | 0.6¹ | 3.0² |
|---|---|---|
| Pre-treatment | 22 ± 11 | 33 ± 7 |
| 24 hrs post administration | 34 ± 14 | 42 ± 10 |
| p | 0.02 | 0.005 |

p = significance by paired t-test;
¹Number of patients = 6;
²Number of patients = 3.

### Example 4

### BAT Antibody Treatment in Conjunction with Stem Cell Transplantation or Donor Leukocyte Infusion—Case Study

Stem cell transplantation (SCT) is a special therapy for patients with cancer or other diseases, which affect the bone marrow. Currently, the major sources of stem cells for transplantation include bone marrow, peripheral blood, and cord blood derived from a variety of donors including the recipient itself (i.e. autologous source) or a donor other than the recipient (i.e. allogeneic source). The three types of allogeneic donors are: syngeneic—in the case where the donor and recipient are identical twins, related—in cases where the donor and recipient are relatives, and unrelated—where the donor is identified through a donor registry or from a cord blood bank. The goal of SCT is to transfuse healthy bone marrow cells into a person after their own unhealthy or dysfunctional bone marrow has been eliminated.

Establishment and repopulation of a normal functioning immune system after transplantation of the new or reinfused cells to the body is a long process. Particularly, the an immune-competent T cell population recovers within 6 to 12 months post transplantation. The time frame for the recovery of an immune response in patients who develop graft versus host disease (GVHD), a common side effect of SCT, is considerably longer. During this time the body is at greater risk for bacterial viral and fungal infections. In addition, the desired therapeutic effect, e.g., anti tumor activity, may be substantially compromised during this recovery period.

Another form of therapy which usually follows SCT failure is donor leukocyte infusion (also termed: DLI) which involves infusion of lymphocytes obtained from blood donated by the original donor. These donated white blood cells contain cells of the immune system that can recognize and destroy cancer cells. The goal of this therapy is to induce a remission of the cancer disease by a process called the graft-versus-tumor effect (GVT). The donor T-cells can attack and control the growth of residual cancer cells providing the GVT effect. Approximately 20 percent of patients treated with DLI develop pancytopenia, a deficiency of all types of blood cells.

BAT antibody treatment is utilized in the aforementioned clinical conditions to promote the recovery of the body's immune system and enhance its return to normal function following SCT or DLI. Administration of BAT concomitant with or following SCT or DLI enhances the generation of disease specific immune cells, in particular NK, T and B cells.

A patient suffering stage IV Hodgkin's lymphoma patient who underwent autologous bone marrow transplantation, received a single dose of humanized BAT antibody (0.6 mg/kg) 4 months after the autologous transplantation. An increase of 80% in the percentage of CD4 T cells 24 hours post humanized BAT antibody treatment was observed. Despite of the patient's advanced condition, stabilization of the disease has been observed for approximately 8 months post treatment with the humanized BAT antibody.

### Example 5

### BAT Antibody Treatment in Conjunction with Chemotherapy and Irradiation—Case Study

Chemotherapy in combination with irradiation is commonly used for the treatment of cancer diseases.

A patient at stage III Chronic Lymphocytic Leukemia received chemotherapy (ESIIAP: etoposide, methylprednisolone, cytarabine, and cisplatin). Approximately 1 month after the chemotherapy the patient was further treated by irradiation and was then treated with a single dose of humanized BAT antibody (0.6 mg/kg). A 40% increase in the percentage of CD4 T cells 24 hours post humanized BAT antibody treatment was observed in this patient. Despite of the patient's advanced condition, stabilization of the disease has been observed for approximately 8 months post humanized BAT antibody treatment.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for the treatment of an immunodeficiency disorder in a subject in need thereof, the method comprising administering a therapeutically effective amount of BAT monoclonal antibody to said subject.

2. The method of claim 1, wherein the immunodeficiency disorder comprises at least one disorder, symptom or abnormality selected from the group consisting of: depletion of lymphocytes, attenuation in the count of lymphocytes, malfunction of lymphocytes and combinations thereof.

3. The method of claim 1, wherein said immunodeficiency disorder is congenital.

4. The method of claim 1, wherein said immunodeficiency disorder is acquired.

5. The method of claim 1, wherein said immunodeficiency disorder is selected from the group consisting of: severe combined immunodeficiency disease, aplastic anemia, acquired immunodeficiency syndrome, X-linked agammaglobulinaemia, common variable immunodeficiency, IgA deficiency, IgG subclass deficiency, Wiskott-Aldrich syndrome, DiGeorge anomaly, Ataxia Telangiectasia, adenosine deaminase deficiency and activation-induced cytidine deaminase deficiency.

6. The method of claim 1, wherein the immunodeficiency disorder is related to viral infection, fungal infection or bacterial infection.

7. The method of claim 1, wherein said immunodeficiency is associated with at least one treatment selected from: chemotherapy, irradiation, transplantation of stem cells and donor leukocyte infusion.

8. The method of claim 1, wherein the immunodeficiency disorder is associated with intoxication.

9. The method of claim 1, wherein the immunodeficiency disorder is associated with aplastic anemia or Myelodysplastic syndromes

10. The method of claim 7, wherein the stem cells are derived from a source selected from the group consisting of: bone marrow, umbilical cord blood and peripheral blood.

11. The method of claim 1, wherein the BAT monoclonal antibody comprises a light chain variable region comprising CDRs selected from the group consisting of: SEQ. ID NO. 13; SEQ. ID NO. 14 and SEQ. ID NO. 15.

12. The method of claim 1, wherein the BAT monoclonal antibody comprises a heavy chain variable region comprising CDRs selected from the group consisting of: SEQ. ID NO. 16; SEQ. ID NO. 17 and SEQ. ID NO. 18.

13. The method of claim 1, wherein the BAT monoclonal antibody is selected from the group consisting of: full length monoclonal antibody, chimeric antibody, humanized antibody, IgG, IgM, IgD, IgA, IgE, diabody, bispecific antibody, linear antibody and fragments thereof.

14. The method of claim 1, wherein the BAT monoclonal antibody is selected from the group of antibody fragments consisting of: Fab, Fab′, F(ab′)₂, Fv; single-chain antibody molecules and multi-specific antibodies formed from antibody fragments.

15. The method of claim 13, wherein the BAT monoclonal antibody is a humanized antibody and wherein the frame regions of the light chain variable region are derived from the light chain variable region of the human TEL9 antibody.

16. The method of claim 15, wherein said frame regions are selected from the group consisting of: SEQ. ID NO. 5; SEQ. ID NO. 6; SEQ. ID NO. 7 and SEQ. ID NO. 8.

17. The method of claim 13, wherein the BAT monoclonal antibody is a humanized antibody and wherein the frame regions of the heavy chain variable region are derived from the heavy chain variable region of the human hsighv1295 antibody.

18. The method of claim 17, wherein said frame regions are selected from the group consisting of: SEQ. ID NO. 9; SEQ. ID NO. 10; SEQ. ID NO. 11 and SEQ. ID NO. 12.

19. The method of claim 2, wherein the immunodeficiency disorder relates to lymphocytes selected from the group consisting of: CD3⁺ cells, CD4⁺ cells, CD8⁺ cells, Thy1.2+ cells, NK cells, NK-T cells, B cells, monocytes and macrophages.

20. The method of claim 1, further comprising administering at least one additional therapeutic agent in combination with a therapeutically effective amount of BAT monoclonal antibody, the at least one additional therapeutic agent being selected from the group consisting of: anti-viral agents, antibiotics, cytokines, T-cell activators, hormones, growth factors, cell vaccines, peptide vaccines, DNA vaccines, antibodies and fragments thereof, T-cell stimulatory antibodies, cell-based therapies, stem cells derived from either the bone marrow, umbilical cord blood, peripheral blood, donor leukocyte infusion.

21. The method of claim 20, wherein the T-cell activator is selected from the group consisting of interleukin-1, interleukin-2, interleukin-4, interleukin-5, interleukin-6, interleukin-7, interleukin-11, interleukin-12, interleukin-13, interleukin-15, interferon-alpha, interferon-gamma, tumor necrosis factors, anti-CD3 antibodies, anti-CD28 antibodies, anti-CTLA4 antibodies, anti-TGF-beta antibodies, anti-4-1BB antibodies, cell-based vaccines peptide vaccines, DNA vaccines, growth factors, phytohemagglutinin, concanavalin-A and phorbol esters.

22. The method of claim 20, wherein said therapeutically effective amount of BAT monoclonal antibody and said at least one therapeutic agent are administered together or sequentially.

23. The method of claim 1, further comprising administering at least one additional anti-cancer agent selected from: anti-metabolic agent, anti-angiogenic agents, cytotoxic agents and anti-tumor therapeutic antibodies and cell based therapies.

24. The method according to claim 1, wherein the subject in need thereof is a human subject.
